(19) United States
(12) Patent Application Publication
(10) Pub. No.: US 2007/0073245 A1
(43) Pub. Date: Mar. 29, 2007

(54) Needle-retractable safety hypodermic syringe

(76) Inventor: Chao-Hua Shih, Wugu Industrial Park (TW)

Correspondence Address: PRO-TECHTOR INTERNATIONAL SERVICES, 20775 NORADA CT., SARATOGA, CA, 95070, US

(21) Appl. No.: 11/177,910

(22) Filed: Jul. 7, 2005

(51) Int. Cl. A61M 5/32; A61M 5/00

(52) U.S. Cl. 604/198; 604/110

(57) ABSTRACT

A needle-retractable safety hypodermic syringe is disclosed to include a barrel, a needle assembly at the front end of the barrel, a plunger mounted with a stopper for expelling medicine out of the barrel through the needle assembly and pulling the needle assembly into the inside of the barrel after the service, a first retaining portion formed inside the rear end of the barrel, and a second retaining portion formed on a connector at the plunger for engaging the fist retaining portion to secure the needle assembly firmly inside the barrel after the service of the safety hypodermic syringe.

### BACKGROUND OF THE INVENTION

1. Field of the Invention

The present invention relates to a safety hypodermic syringe and more particularly, to a needle-retractable safety hypodermic syringe, which allows the needle assembly to be pulled backwards to the inside the barrel and firmly secured in position inside the barrel after the service of the safety hypodermic syringe.

2. Description of the Related Art

A conventional hypodermic syringe is provided with a cap for protecting the needle cannula from injuring a person to prevent a contamination after the service. However, the cap tends to fall from the hypodermic syringe, and the worker may be injured by the needle cannula accidentally, causing a contamination.

In order to eliminate the aforesaid problem, safety hypodermic syringes are developed. Exemplars are seen in Taiwan Patent Application Nos. 92204062 and 93210924. FIGS. 1 and 2 show another prior art safety hypodermic syringe. According to this design, the needle assembly can be pulled backwards with the plunger and received inside the barrel after the service of the safety hypodermic syringe. However, after broken of the plunger from the needle assembly, the needle assembly is movable in the barrel. The needle cannula may be forced out of the barrel to injure a person accidentally.

### SUMMARY OF THE INVENTION

The present invention has been accomplished under the circumstances in view. It is one object of the present invention to provide a needle-retractable safety hypodermic syringe, which allows the needle assembly to be pulled backwards to the inside the barrel and firmly secured in position inside the barrel after the service of the safety hypodermic syringe. To achieve this and other objects of the present invention, the safety hypodermic syringe comprises a barrel, the barrel comprising a hollow cylindrical body, a neck axially forwardly extending from a front end of the hollow cylindrical body, a rear opening in a rear end thereof remote from the neck, and a retaining means disposed at an inside wall of the hollow cylindrical body near the rear opening; a needle assembly, the needle assembly comprising a needle holder fastened to the neck of the barrel, the needle holder having a rear retaining hole in a rear side thereof, and a needle cannula fastened to the needle holder and suspending outside the barrel; and a plunger, the plunger comprising a shank, the shank having a front end and a rear end, a thumb rest formed integral with the rear end of the shank, a connector forwardly extended from the front end of the shank for engaging the retaining hole of the needle holder for enabling the needle assembly to be pulled by the plunger and received inside the barrel, the connector being breakable from the shank when the shank is biased relative to the connector, a retaining means for engaging the retaining means of the barrel to secure the connector and the needle assembly to the inside of the barrel, and a stopper connected to the connector and disposed in contact with an inside wall of the barrel for expelling a medicine out of the barrel through the needle assembly.

### BRIEF DESCRIPTION OF THE DRAWINGS

FIG. 1 is schematic drawing showing a hypodermic syringe according to the prior art.

FIG. 2 shows the needle cannula pierced through the barrel of the hypodermic syringe after the service according to the prior art.

FIG. 3 is an exploded view of a needle-retractable safety hypodermic syringe according to the present invention.

FIG. 4 is a schematic drawing showing the needle assembly moved with the plunger into the inside of the barrel after the service of the needle-retractable safety hypodermic syringe according to the present invention.

FIG. 5 is an enlarged view of a part of FIG. 4, showing the retaining portion of the connector engaged with the retaining portion of the barrel.

FIG. 6 corresponds to FIG. 4, showing the shank of the plunger broken from the connector after the service of the needle-retractable safety hypodermic syringe.

FIG. 7 is a schematic exploded view of an alternate form of the needle-retractable safety hypodermic syringe according to the present invention.

FIG. 8 is a sectional view of a part of the alternate form of the needle-retractable safety hypodermic syringe, showing retaining portion of the connector engaged with the retaining portion of the barrel.

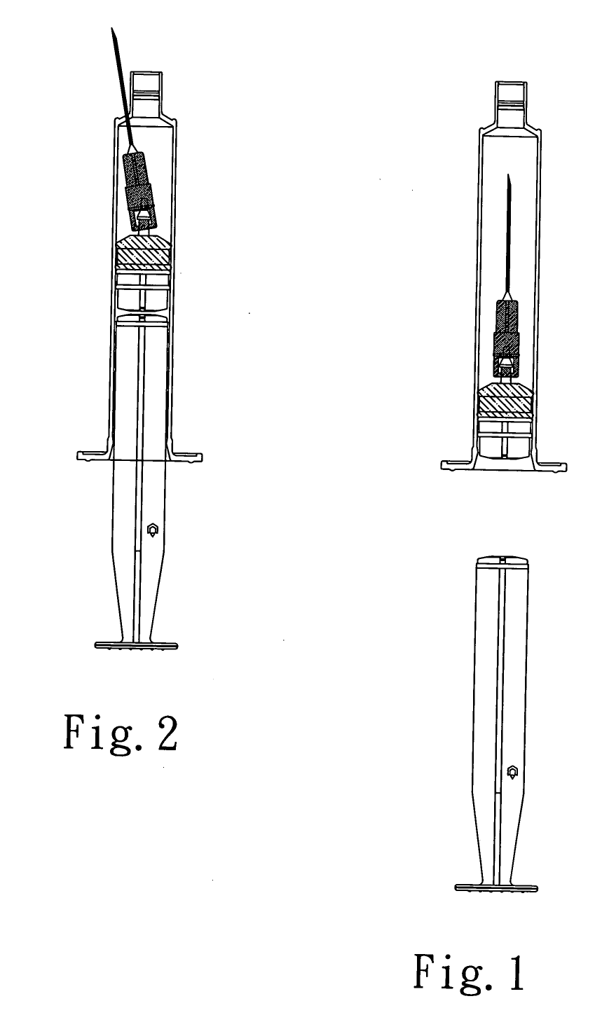

### DETAILED DESCRIPTION OF THE PREFERRED EMBODIMENT

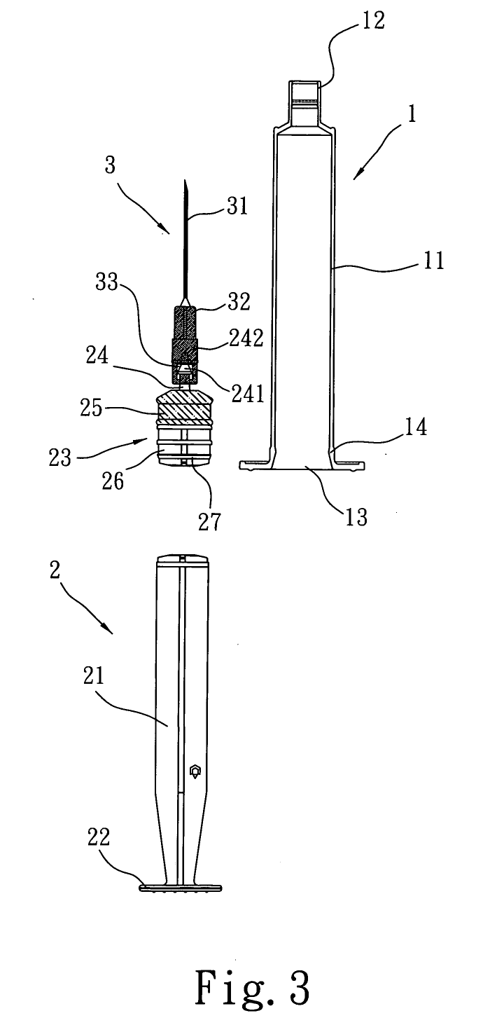

Referring to FIG. 3, a needle-retractable safety hypodermic syringe in accordance with the present invention is shown comprised of a barrel 1, a plunger 2, and a needle assembly 3. After injection, the plunger 2 can be pulled backwards to carry the needle assembly 3 to the inside of the barrel 1, keeping the needle assembly 3 firmly secured to the inside of the barrel 1.

The barrel 1 comprises a hollow cylindrical body 11, a neck 12 axially forwardly extending from one end, namely, the front end of the hollow cylindrical body 11, and a retaining portion (for example, an annular retaining flange) 14 extending around the inside wall of the hollow cylindrical body 11 near the rear opening 13.

The plunger 2 comprises a shank 21, a thumb rest 22 formed integral with one end, namely, the rear end of the shank 21, and a connector 23 connected to the other end, namely, the front end of the shank 21. The connector 23 comprises a front connecting rod 24 insertable into the rear side of the needle assembly 3, a retaining portion (for example, an annular retaining groove) 26 extending around the periphery thereof for receiving the retaining portion (retaining flange) 14 of the barrel 1, and a tapered guide surface 27 disposed at the rear side thereof and adapted to guide the retaining portion (retaining groove) 26 into engagement with the retaining portion (retaining flange) 14 when the user pulled the plunger 2 backwards after the service of the safety hypodermic syringe. The front connecting rod 24 has a pointed piercing tip 242 and a barbed engagement portion 241. The plunger 2 further comprises a rubber stopper 25 fixedly mounted on the connector 23 around the rear side (the foot) of the front connecting rod 24 and movable with the plunger 2 to expel medicine out of the barrel 1 and the needle assembly 3.

The needle assembly 3 comprises a needle holder 32 mounted in the neck 12 of the barrel 1, and a needle cannula 31 fastened to the needle holder 32 and extending out of the neck 12 of the barrel 1. The needle holder 32 has a rear retaining hole 33.

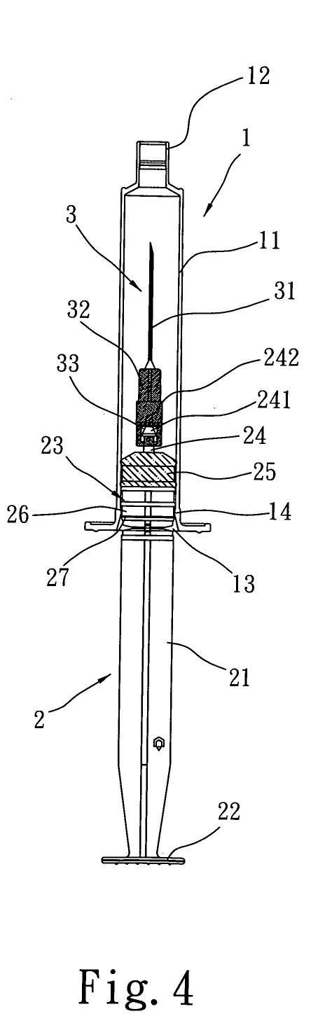

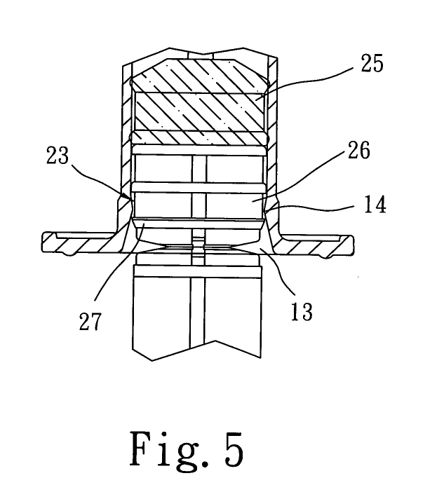

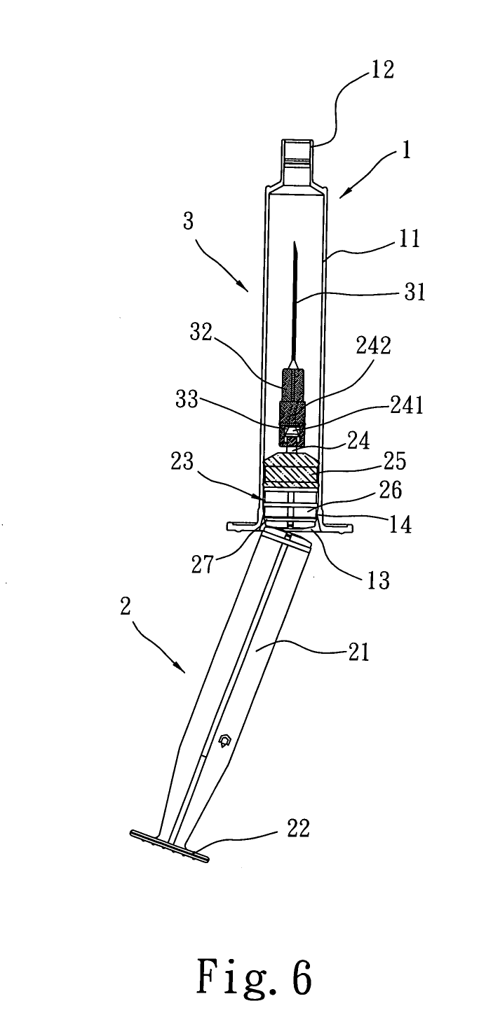

Referring to FIGS. 4˜6, when pushed the plunger 2 forwards in the barrel 1, the rubber stopper 25 is moved with the connector 23 and the shank 21 to expel medicine out of the barrel 1 through the needle assembly 3 into the patient's body, and at the same time the pointed piercing tip 242 is inserted into the needle holder 32 of the needle assembly 3b and the barbed engagement portion 241 is forced into engagement with the rear retaining hole 33 of the needle holder 32. After the injection, the plunger 2 is pulled backwards, and the needle assembly 3 is moved with the connector 23 to the inside of the barrel 1. When pulled the plunger 2 backwards relative to the barrel 1 to the extent, the retaining portion (retaining groove) 26 of the connector 23 is forced into engagement with the retaining portion (retaining flange) 14 of the barrel 1. At this time, the user can bend the plunger 2 to break the shank 21 from the connector 23, keeping the connector 23 with the rubber stopper 25 and the needle assembly 3 firmed secured to the inside of the barrel 1.

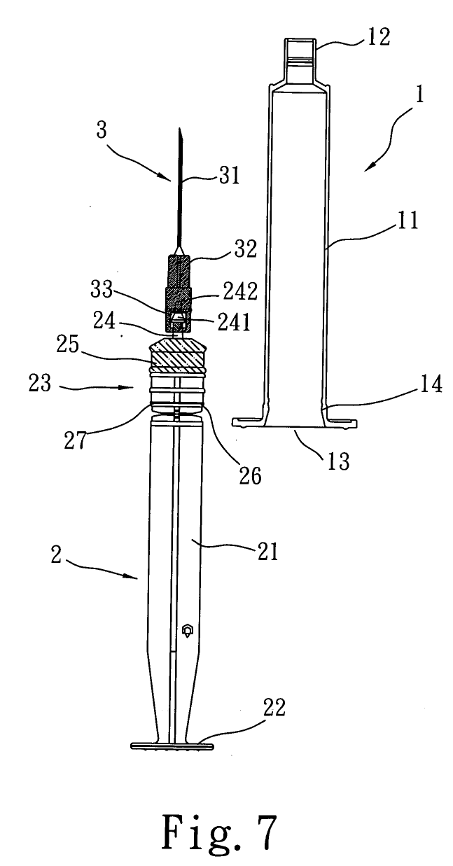

FIGS. 7 and 8 show an alternate form of the present invention. According to this embodiment, the retaining portion 14 of the barrel 1 is an annular retaining groove extending around the inside wall thereof, and the retaining portion 26 of the connector 23 is an annular retaining flange extending around the periphery for engaging the retaining portion (annular retaining groove) 14 of the barrel 1.

A prototype of needle-retractable safety hypodermic syringe has been constructed with the features of FIGS. 3˜8. The needle-retractable safety hypodermic syringe functions smoothly to provide all of the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A safety hypodermic syringe comprising
a barrel, said barrel comprising a hollow cylindrical body, a neck axially forwardly extending from a front end of said hollow cylindrical body, a rear opening in a rear end thereof remote from said neck. and a retaining means disposed at an inside wall of said hollow cylindrical body near said rear opening;
a needle assembly, said needle assembly comprising a needle holder fastened to said neck of said barrel, said needle holder having a rear retaining hole in a rear side thereof, and a needle cannula fastened to said needle holder and suspending outside said barrel; and
a plunger, said plunger comprising a shank, said shank having a front end and a rear end, a thumb rest formed integral with the rear end of said shank, a connector forwardly extended from the front end of said shank for engaging the retaining hole of said needle holder for enabling said needle assembly to be pulled by said plunger and received inside said barrel, said connector being breakable from said shank when said shank is biased relative to said connector, a retaining means for engaging the retaining means of said barrel to secure said connector and said needle assembly to the inside of said barrel, and a stopper connected to said connector and disposed in contact with an inside wall of said barrel for expelling a medicine out of said barrel through said needle assembly.

2. The safety hypodermic syringe as claimed in claim 1, wherein the retaining means of said plunger comprises at least one retaining flange on said connector; the retaining means of said barrel comprises at least one retaining groove for engaging the at least one retaining flange of said plunger.

3. The safety hypodermic syringe as claimed in claim 2, wherein the retaining means of said plunger is an annular retaining flange extending around the periphery of said connector; the retaining means of said barrel is an annular retaining groove extending around an inside wall of said barrel for receiving the annular retaining flange of said plunger

4. The safety hypodermic syringe as claimed in claim 1, wherein the retaining means of said plunger comprises at least one retaining groove on said connector; the retaining means of said barrel comprises at least one retaining flange for engaging the at least one retaining groove of said plunger.

5. The safety hypodermic syringe as claimed in claim 4, wherein the retaining means of said plunger is an annular retaining groove extending around the periphery of said connector; the retaining means of said barrel is an annular retaining flange extending around an inside wall of said barrel for engaging the annular retaining flange of said plunger.

6. The safety hypodermic syringe as claimed in claim 1, wherein said connector comprises a front conehead connecting rod for engaging the retaining hole of said needle holder.

7. The safety hypodermic syringe as claimed in claim 1, wherein the retaining means of said plunger is disposed on said connector.

8. The safety hypodermic syringe as claimed in claim 1, wherein said stopper is formed integral with said connector of said plunger.

9. The safety hypodermic syringe as claimed in claim 1, wherein said stopper is formed of an elastic material.

10. The safety hypodermic syringe as claimed in claim 1, wherein said connector has a tapered guide surface for guiding the retaining means of said plunger into engagement with the retaining means of said barrel.
